(19) United States
(12) Patent Application Publication
(10) Pub. No.: US 2017/0259069 A1
(43) Pub. Date: Sep. 14, 2017

(54) SYSTEM AND METHOD FOR GENERATING PREMODULATED INTERFERENTIAL CURRENTS, PARTICULARLY FOR SPINAL CORD STIMULATION

(71) Applicant: BIOTRONIK SE & CO. KG, BERLIN (DE)

(72) Inventors: MARCELO BARU, TUALATIN, OR (US); DIRK MUESSIG, WEST LINN, OR (US); LARRY STOTTS, TIGARD, OR (US)

(21) Appl. No.: 15/432,996

(22) Filed: Feb. 15, 2017

Related U.S. Application Data
(60) Provisional application No. 62/306,094, filed on Mar. 10, 2016.

(51) Int. Cl. A61N 1/36; A61N 1/05

(52) U.S. Cl. CPC A61N 1/36192; A61N 1/0551; A61N 1/36071; A61N 1/36178; A61N 1/36171; A61N 1/36157

(57) ABSTRACT

A premodulated interferential current, particularly for spinal cord stimulation, is generated using a pulse generator having multiple electrodes. The premodulated current, which is delivered through at least one of the electrodes, includes a train of biphasic pulses having a repetition frequency, wherein each biphasic pulse includes a stimulating phase and a balancing phase. The premodulated current includes an amplitude modulation envelope having an envelope beat frequency smaller than the repetition frequency of the biphasic pulses, wherein the modulation envelope is generated in the pulse generator.

### CROSS-REFERENCE TO RELATED APPLICATIONS

This application claims priority under 35 USC §119(e) to U.S. Provisional Patent Application 62/306,093 filed 10 Mar. 2016, the entirety of which is incorporated by reference herein.

### FIELD OF THE INVENTION

The invention relates to a method and a system for generating premodulated currents, particularly for spinal cord stimulation (SCS).

### BACKGROUND OF THE INVENTION

Tissue stimulation using interferential currents (IFCs) utilizes two independent alternating (e.g., sinusoidal) currents with frequencies in the range of 500 Hz to 20,000 Hz that are injected diagonally of each other, creating an X pattern (see, e.g., U.S. Pat. No. 8,977,363). Such stimulation allegedly provides greater total stimulus intensity where the currents intersect, thereby providing maximum stimulation away from the electrodes rather than adjacent to them, as occurs with traditional bipolar stimulation. However, it is questionable whether true benefits are as significant as those alleged. Since current spread reduces intensity away from the electrodes, even if two currents are superimposed, their total intensity will likely be less than that immediately under the electrodes. Further, the IFC stimulus waveform seen by the targeted nerve fibers depend on their relative orientation with respect to the stimulating electrodes. Thus, the stimulus waveform created by conventional IFC has uncertain results.

Delivering two (or more) stimulus currents concurrently at different sites within a patient's body without crosstalk also presents implementation challenges for an implantable pulse generator (IPG), as all current generators are referenced to the battery of the IPG.

A possible solution is to have two or more entirely separate generators, powered by separate batteries. However, this approach is problematic if an objective is to minimize the size of the implantable pulse generator. U.S. Pat. No. 8,977,363 proposes another solution that has been traditionally used in external electrical stimulators, involving the use of transformer isolation to electrically float the generators. This too adversely affects the size of the system. Stimulation using conventional IFC approaches therefore presents implementation challenges where IPG miniaturization is a goal.

Further, U.S. Pat. No. 8,165,672 describes SCS stimulation using a combination of high frequency (HF) signals and direct current (DC) signals. US Published Patent Application US2014/0257428 describes SCS stimulation patterns using an envelope—HF stimulation—modulation signal.

### SUMMARY OF THE INVENTION

The invention, which is defined by the claims set forth at the end of this document, seeks to reduce the complications of implementing conventional IFC in an implantable pulse generator (IPG). One aspect of the invention involves systems and methods for generating a premodulated interferential current, particularly for spinal cord stimulation (SCS). A pulse generator (particularly an implantable pulse generator, i.e., a pulse generator that is configured to be implanted in a patient) has one or more electrodes, wherein a premodulated current is generated by the pulse generator and delivered using at least two electrodes. The premodulated current includes a train of biphasic pulses having a frequency fₜᵣₐᵢₙ, wherein each biphasic pulse includes a stimulating phase and a succeeding balancing phase.

The biphasic pulses are preferably rectangular pulses, wherein each phase of each biphasic pulse is separated by an interphase delay T_{D} (the interphase delay T_{D} preferably being programmable).

Each balancing phase is preferably followed by an open circuit phase where no current is applied via the electrodes. During the open circuit phase, the electrode potentials are indirectly monitored, and corrected if they exceed a safe voltage window, thereby allowing for uninterrupted therapy.

The pulse generator also preferably generates an amplitude modulation envelope for the premodulated current, wherein the amplitude modulation envelope has an envelope beat frequency f_{beat} smaller than the frequency fₜᵣₐᵢₙ of the biphasic pulses. The modulation envelope of the train preferably ramps up in a linear fashion to a maximal amplitude and then ramps down again in a linear fashion.

The phases of the individual pulses preferably have the same pulse width (PW), and are preferably delivered at a frequency between 500 Hz and 20,000 Hz. The pulses may ramp up and down in amplitude within the modulation envelope; such amplitude variation could be defined, for example, by different programmable indexes. This permits combining the effects of high-frequency and low-frequency nerve fiber stimulation as desired.

It is known that nerves accommodate to a constant signal, tuning out the “electrical massage” effect caused by electrical stimulation to block pain, which is a major complication of traditional tonic-based spinal cord stimulation (SCS). This effect is known as habituation. To prevent this from occurring, the envelope beat frequency f_{beat} is preferably automatically varied by the pulse generator (or manually by the patient, e.g., via a remote control).

The envelope beat frequency f_{beat} can be changed, for example, by automatically removing pulses from the train, and/or automatically adding pulses to the train. Such addition and/or removal is preferably done in a manner that translates into a gradual sweep of the envelope beat frequency f_{beat}, particularly in the form of a triangular pattern between a lower envelope beat frequency f_{beatL} and a higher envelope beat frequency f_{beatH}.

The premodulated currents are preferably delivered simultaneously using multiple electrodes to perform current steering—that is, adjustment of the electrical stimulation field by changing the amplitudes of different current sources—particularly for stimulation “sweet spot” identification.

In order to prevent voltage runaway in the DC blocking capacitors, which are placed in series with the electrodes for protective purposes, and to prevent electrode potential drifts (e.g., accumulated double layer voltages at the electrode-target double layers) into dangerous zones, the balancing phase for the pulses in the train is determined such that each electrode's capacitances (its DC-blocking capacitor and double layer capacitance) charge in the same direction, and the capacitances of the stimulating and return electrodes charge in opposite directions. During the actual electrical stimulation of the target (e.g., during SCS), the electrode potentials (e.g., accumulated double layer voltage) are indirectly monitored, and corrected as required, in order to maintain the electrode potentials within safe windows of operation, thereby better avoiding tissue damage or electrode corrosion. By avoiding voltage runaway and potential drifts, the invention better allows for continuous electrical stimulation of the target, without the need to interrupt stimulation to address these issues.

Preferably, for automatic charge balancing during a stimulation stage in which a premodulated current is applied to a target, stimulating phases and balancing phases for the respective electrode are automatically programmed in a determination stage preceding the stimulation stage. The programming is such that the difference between the balancing current pulse minus the respective stimulation current pulse through the respective electrode satisfy the following conditions:

(1) For one or more stimulating electrodes, the difference equals a positive value (which may be different for different electrodes);

(2) For one or more return electrodes, the difference is positive, and smaller than or equal to the minimum among the difference values for the stimulating electrodes;

(3) For each electrode, both a DC blocking capacitor (Cᵢ, which couples the respective electrode to a current source, a current sink, or a voltage) and a double layer capacitance (C_{dli}, wherein the double layer capacitance is formed by each electrode and its adjacent material) charge in the same direction, whereas the capacitances of the stimulating and return electrodes charge in opposite directions; and

(4) in the stimulation stage, at least one of the electrodes is monitored, and when a voltage (ΔV_{dli}) accumulated at the double layer of the monitored electrode crosses pre-defined thresholds (−ΔV_{AddOCP}, ΔV_{SubOCP}), correction currents (I_{CORRStim}, I_{CORRRet}) are applied to reduce or cancel the accumulated voltages (ΔV_{dli}).

More preferably, for the aforementioned automatic charge balancing during a stimulation stage in which at least one premodulated current is applied to a target, the premodulated current's programmed stimulation currents I_{Ni} and automatically-determined balancing currents I_{Pi} for the respective electrode “i” are programmed in a determination stage preceding the stimulation stage such that the premodulated current results. The difference between the real parts of the respective balancing phase I_{Pi} and the respective stimulating phase I_{Ni}—real I_{Pi}−real I_{Ni} (where “real” indicates the actual value of the circulating current)—through the respective electrode “i” satisfy the following conditions:

(1) For stimulating electrodes, the difference equals an automatically determined positive value I_{Diffi} (which may be different for different electrodes);

(2) For return electrodes, the difference is positive, and smaller than or equal to I_{MinDiff} (where I_{MinDiff} is selected equal to the smallest among the I_{Diffi} values for the stimulating electrodes). This programming ensures that at each electrode “i,” both the DC blocking capacitor (Cᵢ, connected in series with the respective electrode) and the associated double layer capacitance (C_{dli}, formed by the respective electrode with adjacent target material) charge in the same direction, and in opposite directions for stimulating and return electrodes.

(3) In a stimulation stage succeeding the determination stage, a stimulation current pulse followed by a balancing current pulse is repeatedly applied to the target via the stimulating electrode(s) and a return electrode, wherein the pulses are programmed in beforehand in the determination stage. Between a programmed balancing current pulse and the next programmed stimulation current pulse, an open circuit phase is conducted where no current is imposed via the stimulating electrode(s);

(4) In the stimulation stage (particularly during the respective open circuit phase), at least one of the electrodes is indirectly monitored (i.e., without direct current connection). When a voltage (ΔV_{dli}) accumulated at the double layer of the monitored electrode(s) crosses pre-defined thresholds, correction currents are automatically injected that reduce or cancel the accumulated voltages (also including all other electrodes that did not reach a threshold).

Any crossing of the thresholds by the accumulated double layer voltage is preferably automatically detected during therapy delivery by comparing the voltage on the terminal of the DC blocking capacitor opposite the electrode under consideration versus the difference between an internally-generated voltage reference and the estimated accumulated voltage at the DC blocking capacitor.

The current difference (i.e., the necessary charge imbalance) is preferably determined for different patient postures, and/or for different stimulation frequencies. The current difference is preferably determined by letting each stimulating electrode stimulate against a reference electrode in the determination stage, preferably with the reference electrode being defined by the pulse generator's casing.

### BRIEF DESCRIPTION OF THE DRAWINGS

Further versions, features, and advantages of the invention are discussed below with reference to the drawings, wherein:

FIG. 1 shows an exemplary system implementing the invention, the system being particularly adapted for spinal cord stimulation (SCS).

FIGS. 2a and 2b show an exemplary front-end usable in the pulse generator of FIG. 1.

FIG. 3 shows an exemplary premodulated current that might be generated with the invention.

FIG. 4 shows a varying beat frequency that might be generated with the invention.

FIG. 5 shows an exemplary guarded cathode configuration that might be used in the invention.

FIG. 6 shows the potential of a stimulating electrode when an active charge-balanced stimulation protocol is used in a high rate pulsing application.

FIG. 7 shows a schematic representation of an exemplary front-end of an implantable pulse generator usable in the invention.

FIG. 8 shows an example of a stimulation phase and a balance phase of the pulse generator of FIG. 7.

FIG. 9 shows a circuit usable to determine the open circuit potential (OCP).

FIG. 10 shows the circuit of FIG. 9 used to measure the accumulated voltage in cycled electrode “i” during the determination stage connected to the casing of the pulse generator.

FIGS. 11a and 11b show an N to 3 multiplexer (MUX) block for measuring voltages (FIG. 11a) and monitoring electrodes (FIG. 11b).

FIG. 12 shows a stimulation and balance phase following the determination stage in the example described.

FIGS. 13 and 14 show comparators for indirectly monitoring the accumulated double layer voltages.

FIG. 15 shows a correction phase for cancelling accumulated double layer voltages.

FIG. 16 shows a compare phase preceding a correction phase.

FIG. 17 shows a comparator for stopping the injection of a correction phase.

### DETAILED DESCRIPTION OF EXEMPLARY VERSIONS OF THE INVENTION

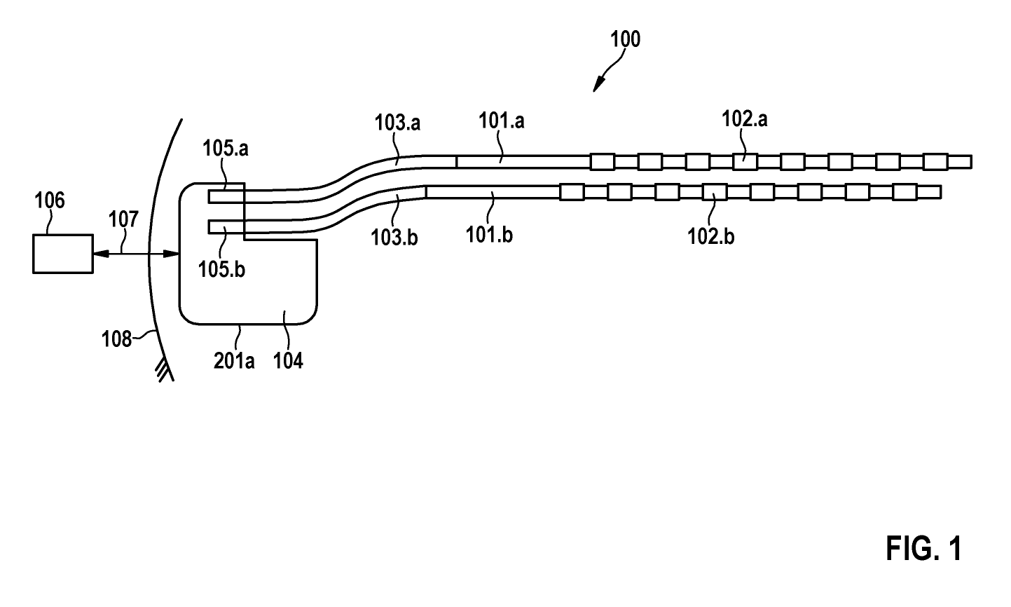

FIG. 1 illustrates an exemplary implantable system 100 for spinal cord stimulation (SCS). The system 100 includes first and second implantable percutaneous leads 101.a and 101.b that are configured to be implanted into a targeted location in the epidural space. These leads 101 may be replaced by paddle leads or other types of SCS leads.

The distal portions of the leads 101.a and 101.b respectively incorporate electrodes 102.a and 102.b, with the leads 101.a and 101.b being shown as octal leads (eight electrodes each). Each electrode 102.a and 102.b is connected to an insulated wire (not shown) that runs inside flexible insulated carriers 103.a and 103.b. During implantation, these carriers 103 get tunneled to the vicinity of the implantable pulse generator (IPG) 104, which is typically implanted subcutaneously in the patient's lower abdominal or gluteal region. The carriers 103.a and 103.b respectively terminate proximally in connectors 105.a and 105.b that are then inserted into the IPG 104 header to allow conduction of electrical charge to the electrodes 102. The IPG 104 case is made of a material that approximates a pseudo reference electrode, such as fractal Ir or TiN, and with an effective area that makes its double-layer capacitance much larger than that of any of the electrodes 102.

The implantable pulse generator (IPG) 104 can communicate with external devices 106, such as a clinician programmer, a patient remote, or an external charger, through suitable radio frequency (RF, e.g. MICS-band) or inductive links 107 that pass through the patient's skin 108. Preferably, an external charger can send power transcutaneously through an inductive link 107 for battery recharge if the IPG 104 is powered by a secondary battery.

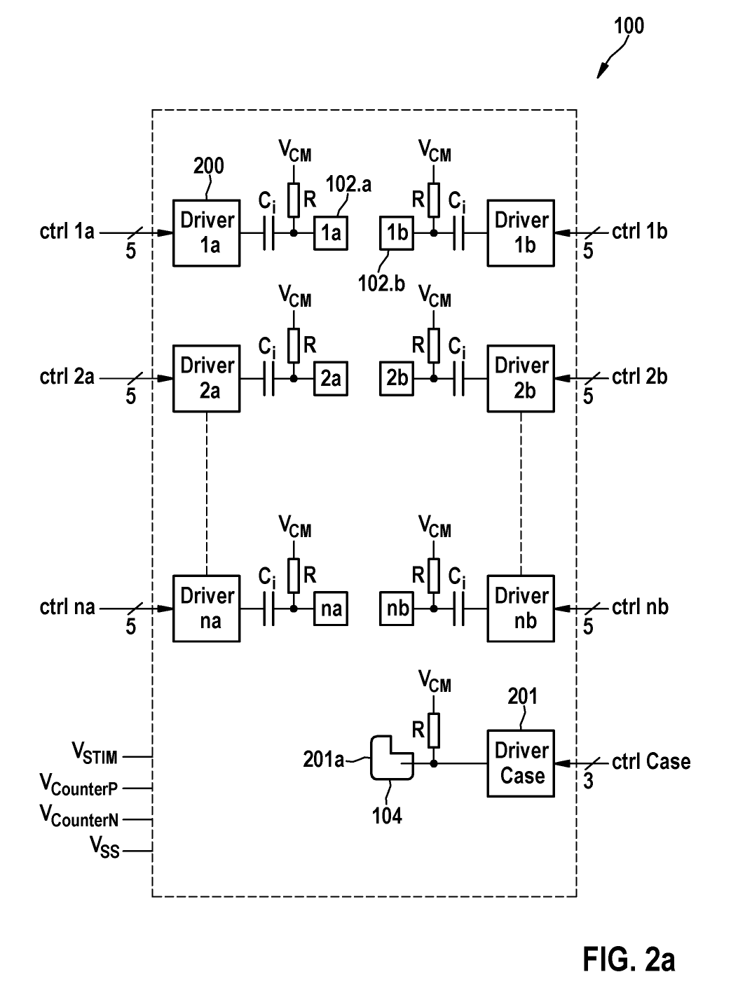

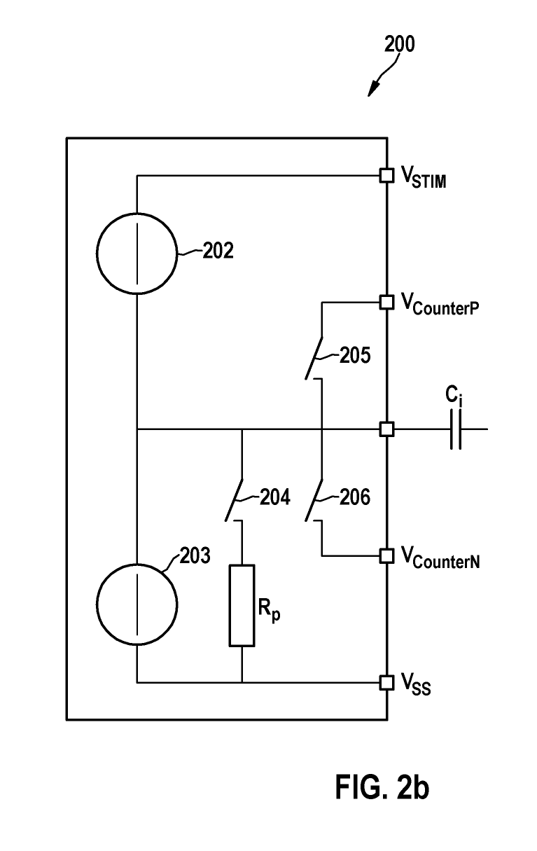

In a preferred version, the IPG 104 has the stimulation front-end shown in FIG. 2A. Electrodes 102.a and 102.b are represented by elements Xₐ and X_{b} (X=1 N) respectively. DC blocking capacitors Cᵢ are provided in series with each electrode Xₐ, X_{b}, and these electrodes can be driven by circuitry in blocks 200. The IPG 104 casing 201a, on the other hand, can be driven by block 201. Resistors R, connected to a common point V_{CM}, are charge bleeding-off resistors.

Each block 200 preferably has five controllable elements as shown in FIG. 2B, where only one block may be active at any time when its respective electrode is utilized for delivery of electrical (e.g., spinal cord) stimulation. Current source 202 permits an electrode 102 to source current from a programmable voltage V_{STIM} (typically up to 16.0 V), whereas current source 203 permits sinking current to V_{SS} (system ground, typically the battery negative voltage) as desired. Having sourcing and sinking currents independently controllable at each electrode 102 permits delivering simultaneous multi-electrode SCS with active charge balancing, thus allowing higher frequency stimulation, and applying current steering to enable targeted stimulation of specific nerve fiber populations. For low frequency applications, analog switch 204 and current limiting resistor R_{P} permit passive charge balancing to minimize power consumption. Although the connection of R_{P} is shown to V_{SS}, other intermediate common potentials may be utilized for passive charge balancing.

For active charge balancing, analog switches 205 and 206 permit currents to circulate from voltage V_{CounterP} or to voltage V_{CounterN} respectively. Typically, V_{CounterP} will be close to V_{STIM} while V_{CounterN} will be close to V_{SS}. In some cases, depending on the impedance and programmed stimulation current, V_{CounterN} and V_{CounterP} need to be offset up to 2.0 V from V_{STIM} or V_{SS} to prevent the circuitry in blocks 200 from exceeding V_{STIM} or going below V_{SS}, which would trigger undesired parasitic conduction of solid-state elements in these blocks 200.

The IPG 104 case driver 201, on the other hand, only needs to include the analog switches 204 . . . 206 and the current limiting resistor R_{P}.

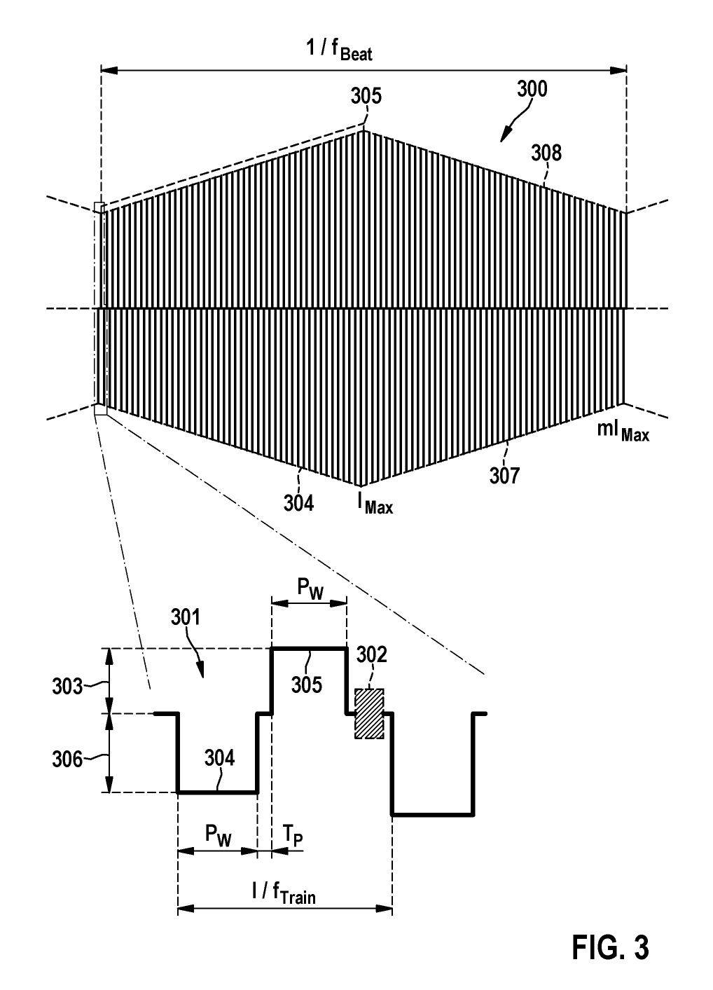

In a preferred version of the system 100 of FIG. 1, two electrodes 102 from the same lead 101, e.g., 3a and 4a from lead 101.a, or an electrode 102 and the IPG 104 case, are used for stimulation using a premodulated current 300 generated inside the IPG 104 as shown in FIG. 3. Such a premodulated current can be preferably defined by the user via an external device (e.g., a clinician programmer) by specifying the following parameters: (1) the frequency of a train of biphasic pulses f_{Train} (equivalent to the sinusoidal carrier frequency in conventional interferential current IFC stimulation); (2) the maximum stimulation amplitude I_{MAX}; (3) the amplitude modulation index m; and (4) the envelope beat frequency f_{Beat}. Each of these programmable parameters will now be discussed in further detail in turn.

The pulse frequency f_{Train} is preferably programmable in the range of 500 Hz to at least up to 16,384 Hz, most preferably up to 20,000 Hz. Each biphasic pulse 301 preferably has identical duration of its phases (a stimulation phase 304 and a balancing phase 305), which have a pulse width (PW) programmable between 10 μs to 1,000 μs. The two phases 304 and 305 are preferably separated by a programmable interphase delay T_{D} in the range of 10 μs to 100 μs. For example, if f_{Train} is programmed equal to 4,000 Hz (i.e., a 250 μs period), PW and T_{D} can be set to 110 μs and 10 μs respectively, which leaves up to 20 μs for an open circuit phase 302. This open circuit phase 302 preferably follows the end of the second phase 305 of each biphasic pulse 301 and is preferably used to indirectly monitor the electrode potentials and correct them (as described below) if they exceed a safe voltage window (typically ±100 mV).

As described above, the programmable pulse frequency f_{Train} limits pulse width PW. Pulse width PW in turn limits the maximum stimulation amplitude I_{Max}, as the charge injected per pulse should not exceed the maximum allowable charge injection (which is typically on the order of 10 μC for SCS). Given this constraint, the maximum stimulation amplitude I_{Max} is typically programmable up to 25 mA

The amplitude modulation can be programmed on or off, and when programmed on, the amplitude modulation index m can be preferably programmed in eight discrete steps from 0.125 to 1.000. FIG. 3 shows an index m equal to 0.500 (i.e. 50% modulation).

The number of pulses in each ramp up (and down) is preferably in the range of 16 to 128. Together with parameter f_{Train}, this number determines the possible programmable envelope beat frequencies f_{Beat}. Using the foregoing example, with 1/f_{Train} set at 250 μs (i.e. 4,000 Hz), 1/f_{Beat} can have programmable values in the range of approximately 8 ms to 64 ms. Thus, in this example, the envelope beat frequency f_{Beat} can be in the range of traditional tonic stimulation frequencies (typically 40 Hz, a 25 ms period).

Preferably, for a given stimulation phase 304, the stimulation control logic in the IPG 104 automatically determines the amplitude 303 of the balance phase 305 of each pulse 301 in the train 300 by applying a determination stage methodology described below. In this case, the M determination pulses are “balanced as-programmed ramping pulses”. Since Faradaic charge transfer is typically required to elicit a physiological response via electrical stimulation, the envelope of the balancing phases 305 may have lower amplitude than that of the pulses in the stimulation phase 304, as the determination stage may define unbalanced biphasic stimulation, with the amplitude 303 of the balancing phase 305 being less than the amplitude 306 of the stimulating phase 304. During the actual delivery of electrical stimulation to the target nerve(s) in the stimulation stage 304, indirect monitoring of the electrode potentials and corresponding correction (as required) occurs during the open circuit phase 302, as will be described below.

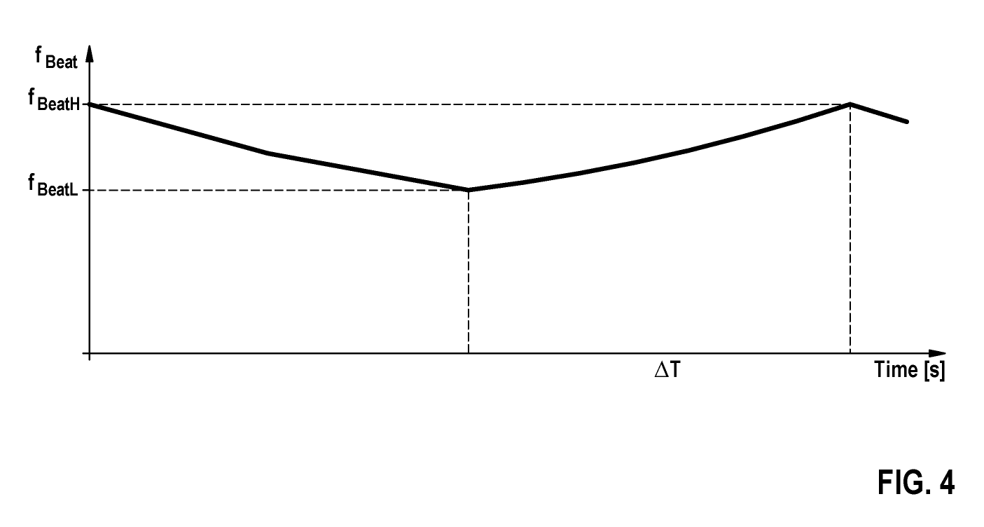

A preferred option is to have the control logic in the IPG 104, or have the patient (by use of a remote control), automatically sweep the envelope beat frequency f_{Beat} in time between two limits f_{BeatH} and f_{BeatL} to deter habituation to stimulation. FIG. 4 shows an exemplary triangular sweep pattern. Such a sweep can be achieved by adding or removing pulses 301 in consecutive envelope beat periods and during a time ΔT. In the foregoing example (i.e., 1/f_{Train} equal to 250 μs), is it possible to inject pulses 301, e.g., 42 to 62 pulses 301, to generate f_{Beat} frequencies that vary between a f_{BeatH} and f_{BeatL} equal to approximately 48 Hz and 32 Hz respectively. This provides a ±20% jitter around the traditional 40 Hz default tonic SCS frequency. The ΔT parameter may be programmable in the range of 1.0 s to 6.0 s. An alternative sweeping pattern (not shown) is a rectangular sweep where f_{Beat} periodically changes discretely between f_{BeatH} and f_{BeatL} without going through intermediate frequencies.

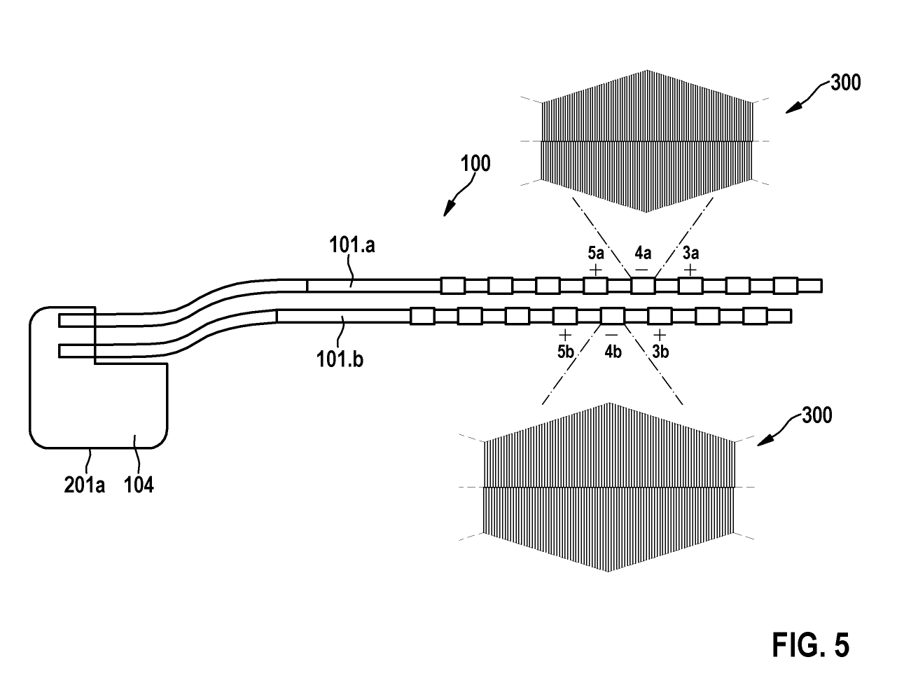

In another preferred version, more than one premodulated current 300 is delivered simultaneously allowing for current steering stimulation. A preferred guarded cathode configuration is shown in FIG. 5. Electrodes 3a, 3b and 5a, 5b of leads 101.a and 101.b operate as anodes whereas electrodes 4a, 4b of both leads 101.a, 101.b operate as cathodes for delivering electrical stimulation, i.e. the currents 300. Different weights to the stimulating currents (i.e. fractional currents) 300 can be programmed through electrodes 4a, 4b to permit targeting more central points of stimulation on the dorsal column.

The invention therefore permits delivering simultaneous, multi-electrode stimulation with similar effects to conventional interferential currents (IFC), but without modifying the architecture of the implantable pulse generator (IPG) 104 designed to deliver either low or high frequency pulsed stimulation.

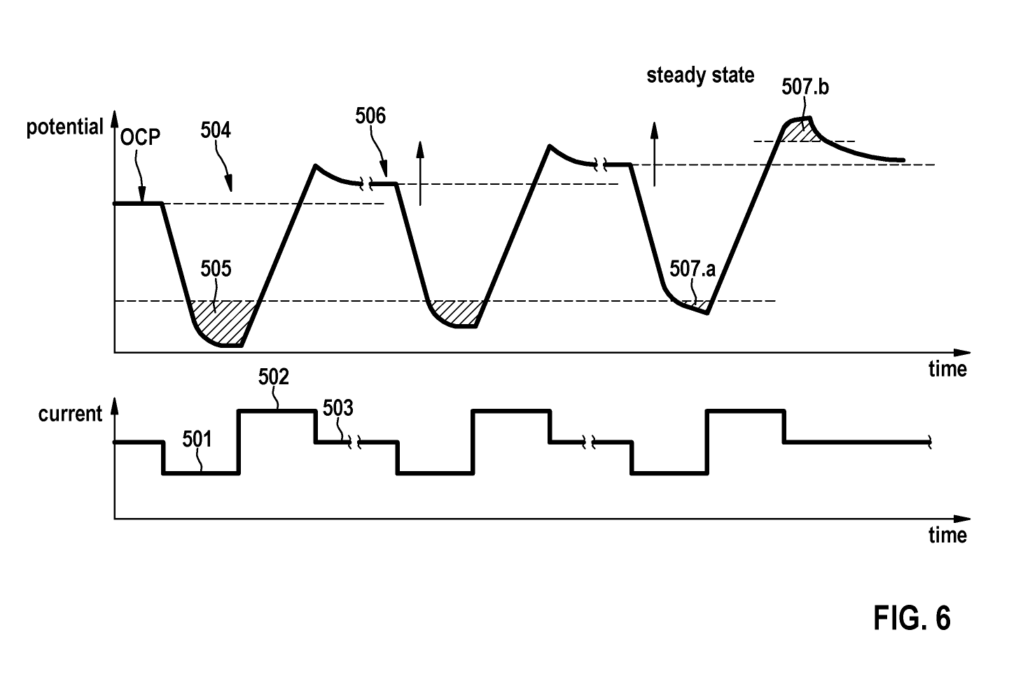

Regarding the automated charge balancing discussed above, FIG. 6 shows in a general manner the potential of a stimulating electrode when an active charge-balanced stimulation protocol is used in a high rate pulsing application. Here the stimulation pulse consists of a stimulation phase 501 (cathodic pulse) and a balancing phase 502 (anodic pulse), followed by an open circuit phase 503 where no current is imposed by the pulse generator 104.

As seen in FIG. 6, the electrode potential begins from its open circuit potential (OCP) (measured against a suitable voltage reference electrode). During delivery of the first cathodic pulse 504, the double layer at the electrode-tissue interface reversibly charges and the electrode may begin to transfer charge into Faradaic reactions 505 as its potential moves negative. Since it is likely some irreversible charge transfer will occur during the stimulation phase 501, not all of the injected charge may go into charging the double layer. Hence, only a fraction of the cathodic charge of pulse 504 would be required during the anodic phase 502 to bring the potential back to OCP. If the anodic pulse 502 is instead fully balanced with the cathodic one 501, as classically implemented in IPGs, the pre-pulse s potential 506 of successive pulses moves positively until the same amount of charge is lost during the cathodic and anodic phases (shaded areas 507.a and 507.b). If this occurs, the anodic Faradaic reaction 507.b may cause electrode corrosion. In the case of a platinum (Pt) electrode, for example, Pt oxide (PtO) may be formed, and soluble Pt compounds—such as cisplatin [PtCl₂(NH₃)₂], which is toxic—may be generated when such PtO reacts in the chloride medium.

The system 100 is therefore preferably configured to deliver stimulation in a manner that automatically adjusts the charges injected to maintain safe operation, and to prevent voltage runaway in the DC blocking capacitors Cᵢ (i denoting one of the electrodes).

During charge-imbalanced stimulation, the shift in pre-pulse potential may be either positive or negative with respect to the open circuit potential (OCP) depending on the amount of imbalance. To monitor electrode voltage drift and compensate for it during therapy (without interruption), the system 100 delivers the minimum charge imbalance necessary to guarantee that at each active electrode, both its associated DC blocking capacitor Cᵢ and double layer (which are in series) charge in the same direction. In the system 100, the stimulating electrodes will charge in one direction whereas the return electrodes will charge in the opposite direction to provide compensation when certain voltage limits are reached.

The determination of the necessary imbalance may be performed prior to electrical stimulation of the target, for different patient postures, and depending on the stimulation frequency, by first independently cycling through each stimulating electrode to be used for electrical stimulation, and stimulating (as programmed for electrical stimulation) against a pseudo reference electrode instead. Such a pseudo reference may be the IPG casing 201a. The system 100 then cycles through all return electrodes except one, which is forced to handle the current mismatches. During this “determination stage,” parameters that measure the final “unbalance” for each active electrode are saved, and the stimulation and return electrodes with the largest voltage drift, as well as the forced return electrode, are selected for indirect monitoring during the actual electrical stimulation of the target (the patient).

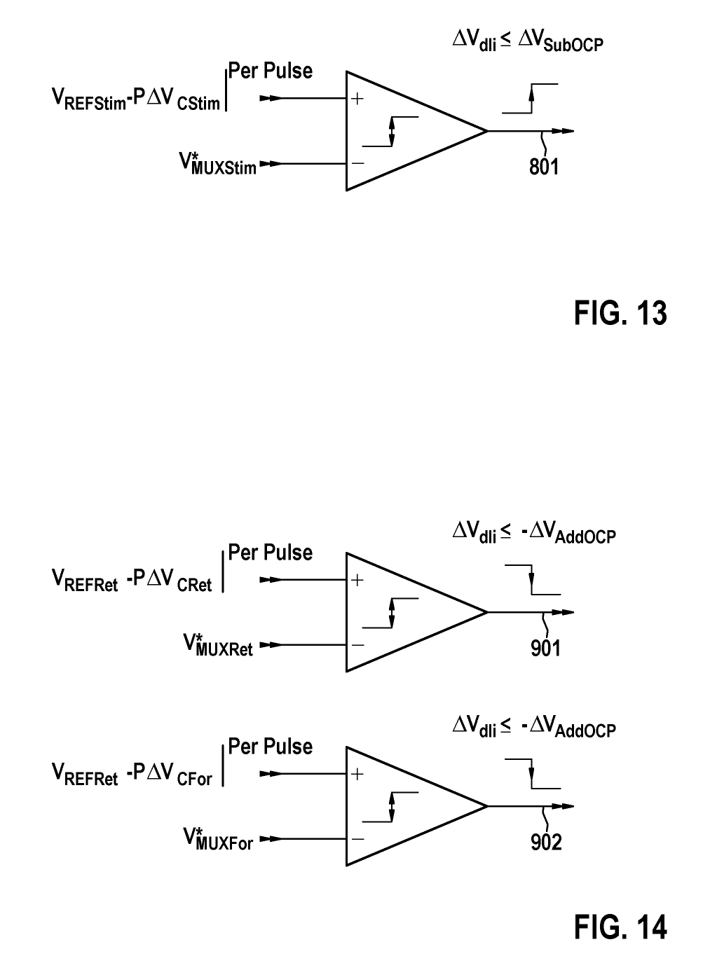

Once the determination stage is completed, electrical stimulation of the target is delivered as programmed During the open circuit phases, the accumulated electrode-tissue double-layer voltages of the electrodes selected for monitoring are indirectly compared against variable reference voltages internally generated in the IPG 104. These comparators (examples shown in FIGS. 13-14, discussed below) preferably allow monitoring the stimulating and return electrode voltages with the largest excursions, and the forced return electrode, between programmable limits without directly accessing such electrode voltages. In particular, there are no measurements during electrical stimulation of the target, and rather there are only comparison of voltages (e.g., a minimum of three voltages on the other side of the DC blocking capacitors for the selected electrodes) that indirectly assess the double-layer voltages accumulated. Advantageously, this approach reduces power consumption (as no amplifiers are used during the actual electrical stimulation of the target), minimizes time to decide on the status of the electrodes, and requires no DC path from an electrode.

Preferably, once a comparator triggers, correction phases take place to start moving the accumulated charges in the opposite directions. These correction phases can either be performed by having a separate active phase during part of the open circuit phases or by adjusting successive balance phases.

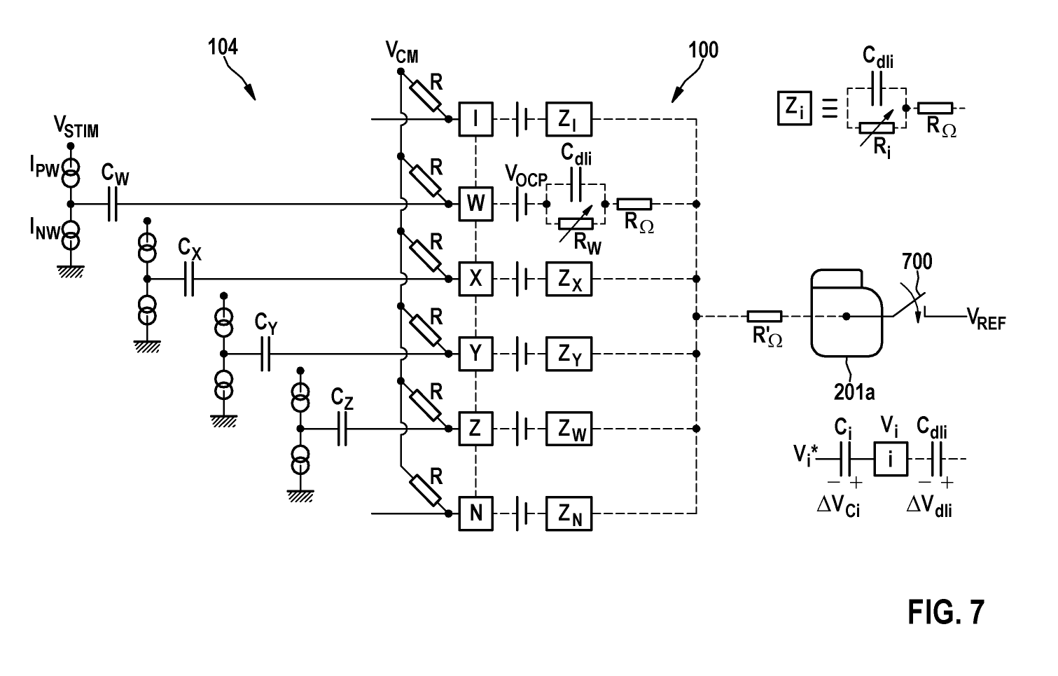

An exemplary method for automated charge balancing will now be described. FIG. 7 shows a schematic representation of a front-end of an implantable pulse generator (IPG) 104, such as an IPG of a spinal cord stimulator (SCS). As schematically depicted at the top right of FIG. 7, each electrode-tissue interface is modeled by an impedance Z, which includes the electrode-tissue double-layer capacitance C_{dli} in parallel with a variable resistor Rᵢ (representative of Faradaic reactions that may occur during stimulation/balancing), in series with R_{Ω}, which represents the ohmic drop of the tissue electrolyte in the vicinity of an electrode.

The IPG case 201a is preferably made of a material that approximates a pseudo reference electrode (e.g., fractal Ir or TiN) and may include an effective area that makes its double-layer capacitance C_{Case} (not shown) much larger than C_{dli} (i=1 N). The electrodes can be made, for example, of Pt, Pt/Ir, or fractal Ir. The open circuit potential (OCP) V_{OCP} shown in FIG. 7 is defined with respect to the IPG case 201a when the latter is connected to an internally-generated voltage reference V_{REF} via switch 700. Since all electrodes are of the same material and have similar areas, it can be considered they all have the same Voce as reflected in FIG. 7.

A similar R′_{Ω} represents the ohmic drop in the vicinity of the IPG case 201a. The R_{Ω} and R′_{Ω} actual values can be neglected for the purpose of this analysis, as voltage monitoring for safe operation particularly occurs during the open circuit phases 503 when no current is imposed by the IPG 104, and thus their actual values are irrelevant. The voltage V_{STIM} in FIG. 7 is preferably programmed with the required minimum overhead for steady-state stimulation.

Cᵢ represents each DC blocking capacitor associated with each electrode (i=1 N, with only C_{W} to C_{Z} shown in FIG. 7), which are nominally all equal. It can also be assumed that Cease, by design, is much larger than Cᵢ. Typically, the Cᵢ value used in IPGs is in the order of 10 μF. C_{dli}, on the other hand, has (for example) a value on the order of 12.5 μF, assuming an SCS Pt electrode. Fractal Ir coated electrodes will present a higher C_{dli}; in the case of nerve cuff electrodes, C_{dli} may be lower. However, the invention assumes no particular relative values between C_{dli} and Cᵢ for the purpose of implementing safe electrical stimulation.

Components R in FIG. 7 are bleeding resistors (e.g., hundreds of kΩ), placed in star configuration, as typically utilized in IPG front-ends for passive charge neutrality. The invention preferably re-utilizes the resistors for the purpose of implementing safe stimulation, as discussed below.

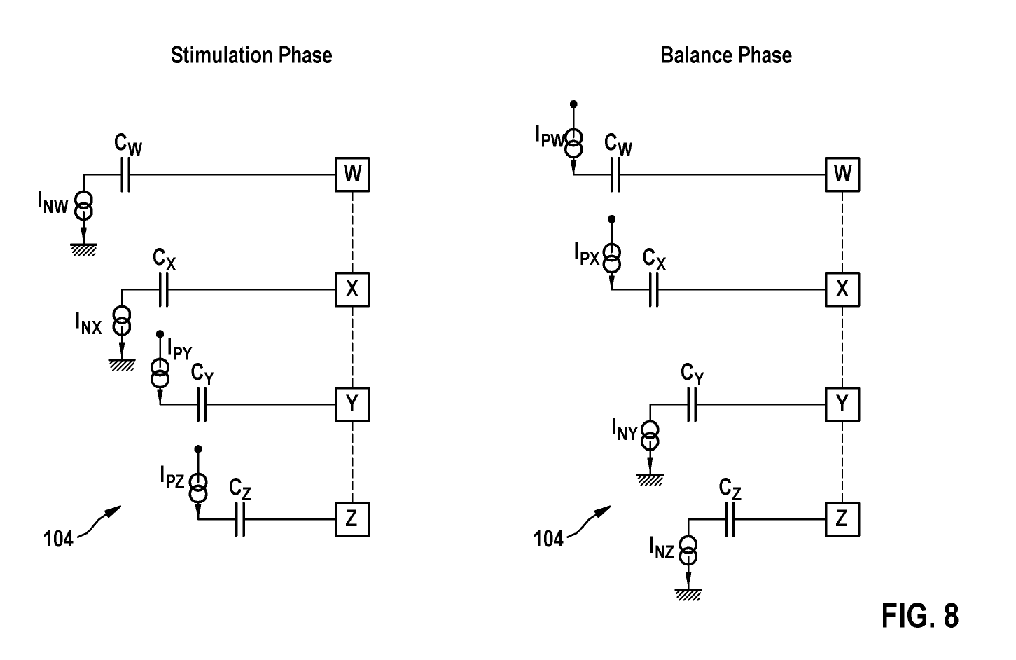

Assume that electrodes W, X, Y, Z are active during delivery of electrical stimulation to the target, and that (for example) W, X are the stimulating electrodes of the stimulation phases, and Y, Z are the return electrodes, as shown in FIG. 8. During the stimulation phase, sinking currents I_{NW} and I_{NX} will flow through electrodes W and X respectively, whereas sourcing currents I_{PY} and I_{PZ} flow through electrodes Y and Z. The currents are programmed so the total cathodic current equals the total anodic current:

I_{NW}+I_{NX}=I_{PY}+I_{PZ} (1)

Assuming the sourcing currents (those from V_{STIM}) present larger output impedance than the sinking currents (those to ground), the latter will accommodate their real values to satisfy eq. (1). Preferably, the invention adjusts the output impedance of the current source associated with at least one of the return electrodes in the stimulation phase (e.g., contact Z) to implement safe operation, as described below.

An active balance phase provides the opposite arrangement as shown in FIG. 8, i.e. currents I_{PW} and I_{PX} flow instead through electrodes W and X respectively, whereas currents I_{NY} and I_{NZ} will flow through electrodes Y and Z.

For the actual electrical stimulation (therapy) of the target, currents I_{NW}, I_{NX}, I_{PY}, and I_{PZ}, the stimulation phase pulse width (PW_{Stim}, common to all), the balance phase pulse width (PW_{Bal}, common to all), the interphase delay T_{D} (i.e. the time between the end of a stimulation pulse and the start of the associated balancing pulse), and the stimulation frequency are preferably selectable and programmable in an IPG 104. For high pulsing rates, and for closed-loop neurostimulation based on neural response, PW_{Bal} is preferably selected equal to PW_{Stim}, with both being programmed as a single parameter pulse width (PW). The balance phase currents I_{PW}, I_{PX}, I_{NY}, and I_{NZ} can be the unknowns the system may automatically determine and adjust to implement safe stimulation without therapy interruption.

For safe tissue and electrode stimulation, the accumulated voltage of the equivalent double-layer capacitances (ΔV_{dli} where i=W, X, Y, Z in the example) should remain within a safe window. With the sign shown in FIG. 7 (bottom right), this translates into

−ΔV_{AddOCP}≦Δv_{dli}≦ΔV_{SubOCP} (2)

where ΔV_{SubOCP} and ΔV_{AddOCP} respectively limit the excursion of the electrode voltage in the negative and positive directions with respect to its open circuit potential (OCP). The limit values may be determined via in-vitro experiments using a suitable electrolyte, confirmed in-vivo, and programmed in the IPG 104. Preferably, the window is symmetrical and a few hundred mV wide (e.g. ±100 mV).

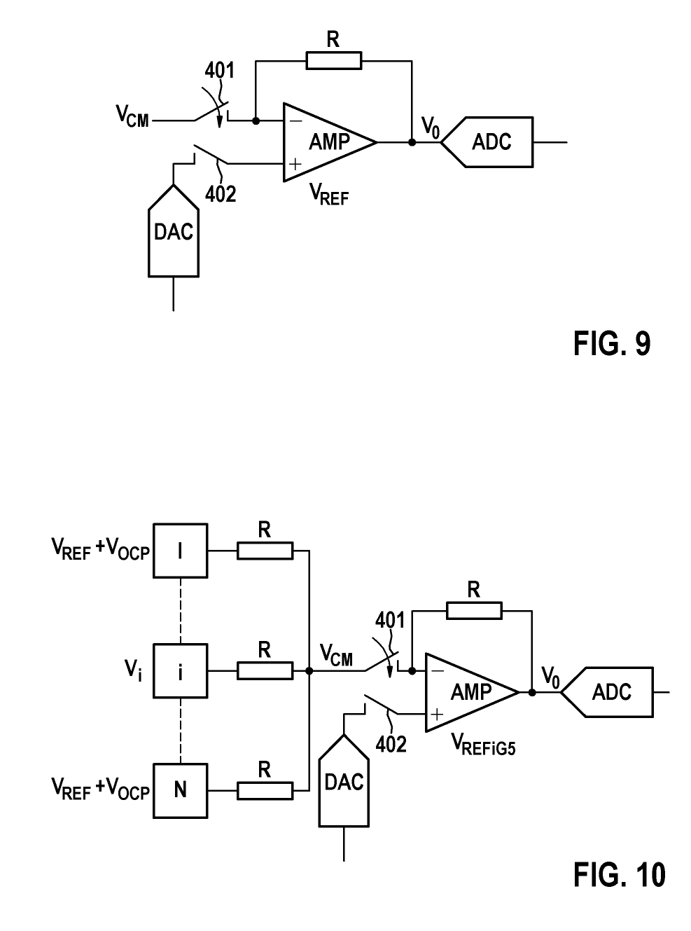

A preferred arrangement for safe stimulation is the following: prior to delivery of the actual electrical stimulation to the target and particularly for different patient postures, the IPG 104 first estimates V_{OCP}. To do so, it is configured to measure the common point V_{CM} of the bleeding resistor network R (see FIG. 7), preferably via the circuit of FIG. 9 (switches 401 and 402 are closed) when the digital-to-analog converter block (DAC) outputs a reference voltage V_{REF} and the IPG case 201 is connected to this voltage. In this case, the output Vo of the amplifier AMP equals

Vo=−NV_{OCP}+V_{REF} (3)

which is preferably digitized via the analog-to-digital converter block (ADC). The V_{OCP} is then calculated and stored in the IPG 104; N is typically 2, 4, 8, 16, or 32 in a neurostimulator, so digital division is straightforward. Switches 401 and 402 are particularly designed with negligible charge injection and on-resistance compared to R. The amplifier AMP offset is also negligible for the purpose of determining V_{OCP}. The resistor R in the feedback of amplifier AMP is preferably matched with the resistors R of FIG. 7 to reduce measurement error.

As previously mentioned, to be able to monitor voltage drift and compensate for it, the system 100 is preferably configured to deliver the minimum charge imbalance that guarantees (at each electrode) that both Cᵢ and C_{dli} charge in the same direction. The stimulating electrodes (W and X in the example) and the return electrodes (Y and Z in the example) of the stimulation phase will charge in opposite directions to allow compensating once a limit given by condition (2) is reached.

Prior to the stimulation stage (where the actual electrical stimulation of the target takes place), the determination stage may proceed as follows:

The system 100 preferably first cycles through each stimulating electrode independently (W and X in the example), and injects M (M=2, 4, 8, . . . ) “balanced as-programmed pulses” leading to the ramp envelope (i.e. I_{Pi} is automatically programmed equal to I_{Ni}) against the IPG case 201a (the return electrode in the determination stage). The balance will then only be limited by the current matching between the real I_{Ni} and real I_{Pi}, which is typically calibrated for and a few percent apart. M may be selected to improve accuracy of the calculations detailed below. In between the cycling of electrodes W and X (in the example), a complete passive balance phase for electrode W and IPG case 201a (with hardware not shown in FIG. 7) is preferably performed to guarantee an electrical-neutral system before cycling the next electrode (X in the example). The same procedure applies when two or more stimulating electrodes are utilized instead for therapy.

For the determination stage, V_{STIM} may be re-programmed with different values to mimic the actual varying voltage that will appear across each current source/sink during therapy. For electrode W, for example, V_{STIM} may be temporarily re-programmed during the determination stage with a value equal to

V_{DSn}+R_{W2casemax}*I_{NWMax}(I_{NWMax}*PW)/C_{WdlVCmin}

where V_{DSn} is a “safe” compliance voltage required for the current sinks to operate, R_{W2casemax} is the measured impedance between electrode W and the IPG case 201a increased by the measurement error, I_{NWmax} is the stimulation current through electrode W increased by the allowable error, PW is the stimulation pulse width, and C_{WdlVCmin} is the measured series capacitor C_{W}, C_{dlw} decreased by the measurement error. It is assumed that V_{DSn} has enough overhead to accommodate the maximum steady-state accumulated voltage on C_{W} and C_{dlw} for the determination stage to properly operate under such reduced V_{STIM}. Given each electrode is much smaller than the IPG case 201a, this setup emulates what each electrode will see under a multi-current therapy setup.

After the M determination pulses in the stimulating electrode “i” (i=W or X in the example), connecting again the circuit of FIG. 9 and the IPG case 201a results in the circuit of FIG. 10. All electrodes, except the cycled “i” (which was active), still present a voltage equal to (V_{REF}+V_{OCP}). Programming the DAC block reference to a voltage V_{REFFIG5} equal to (V_{REF}+V_{OCP}) results in

Vo=ΔV_{dli}+V_{REFFIG5} (4)

as

Vᵢ=−ΔV_{dli}+V_{REF}+V_{OCP}=−ΔV_{dli}+V_{REFFIG5}(5)

(see FIG. 7 for the defined sign of ΔV_{dli}).

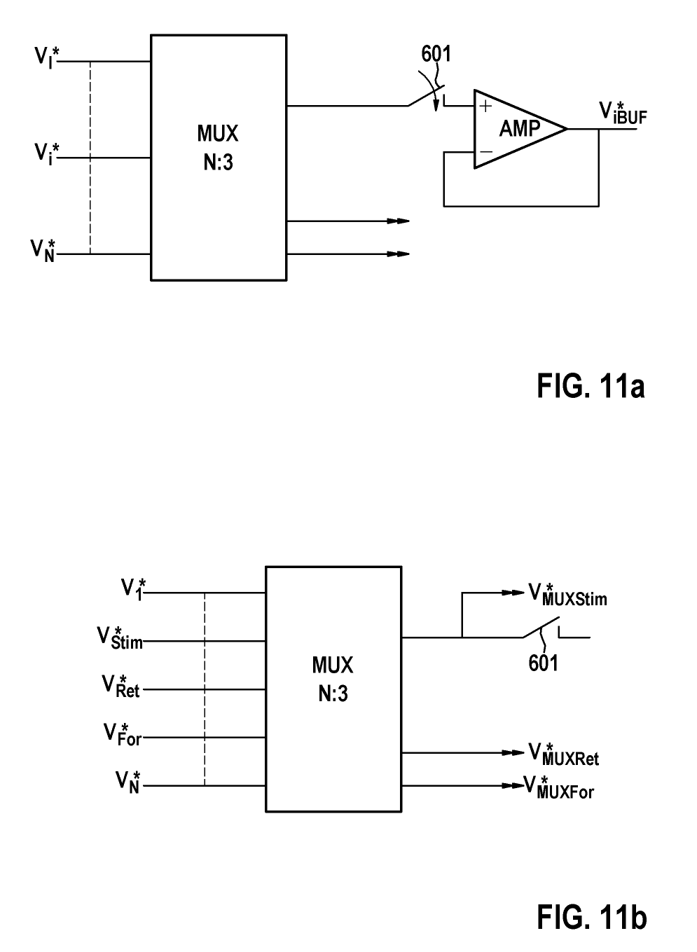

At the same time, the system 100 also measures V*ᵢ, which is the voltage at the other terminal of the DC blocking capacitor Cᵢ of the cycled active electrode “i” (see FIG. 7 bottom right). This is measured via the N to 3 multiplexer (MUX) block, switch 601 and buffer (AMP) shown in FIG. 11a (V*_{iBUF} is the output signal).

From Vᵢ determined above (see eq. (5)) and V_{iBUF}, the accumulated voltage ΔV_{Ci} (from current mismatches) on the blocking capacitor Cᵢ can be calculated as (Vi−V*_{iBUF}) (see FIG. 7 for the sign of ΔV_{Ci} as measured).

If both ΔV_{dli} and ΔV_{Ci} are positive, the balance phase for the cycled electrode “i” can be left as programmed for the determination stage. No adjustments are necessary as the positive voltages indicate the mismatch in the real I_{Ni} and real I_{Pi} is causing the balancing charge to be less than the stimulation charge. The misbalance current I_{Diffi}, i.e. real I_{Ni}−real I_{Pi}, can be estimated to be at least

I_{Diffi}=Cᵢₘᵢₙ(ΔV_{Ci})/(MPW)(i=W or X in the example) (6)

where Cᵢₘᵢₙ is the minimum value of the DC blocking capacitor Cᵢ, ΔV_{Ci} is the measured accumulated voltage, and PW is the programmed pulse width.

On the other hand, if ΔV_{dli} is negative, this implies the electrode “i” potential would be moving positively pulse after pulse, so less balancing charge is required to avoid this situation. Preferably, the balancing charge reduction is determined as follows.

A prior impedance measurement allows estimating C_{dli} for the electrode “i” under consideration (either W or X in the example), with a certain error. Thus, the current I_{Lessi} to be subtracted from the automatically selected I_{Pi} can be calculated as:

I_{Lessi}=C_{dlimax}(−ΔV_{dli})/(MPW)(i=W or X or none in the example) (7)

where C_{dlimax} is the measured C_{dli} with the maximum added error, ΔV_{dli} is the accumulated double-layer voltage (see FIG. 7), and PW is the programmed pulse width as defined before.

A lookup table can be implemented in the IPG 104 to determine each I_{Diffi}, I_{Lessi} based on the corresponding C, ΔV, and (M PW).

For those electrodes with negative ΔV_{dli}, I_{Pi} will then be automatically re-programmed equal to

new I_{Pi}=old I_{Pi}−I_{Lessi}=W or X or none in the example) (8)

where I_{Lessi} is the current estimated above.

Having a positive ΔV_{dli} and a negative ΔV_{Ci} is not possible, as the latter implies the automatically programmed I_{Pi} was larger than the selected I_{Ni} (by mismatch), which will always result in a negative ΔV_{dli} regardless of whether Faradaic reactions were present or not during the stimulation phase.

After initially cycling through all stimulating electrodes, a new set of M pulses, with the modified balance phase, is preferably injected for the stimulating electrodes that required I_{Pi} adjustment. Their new I_{Diffi} is then estimated and stored, and it is confirmed that both Cᵢ and C_{dli} accumulated charge in the same direction.

At the end of this process, all stimulating electrodes “i” (W and X in the example) will in theory satisfy

real I_{Pi}=real I_{Ni}−I_{Diffi} (9)

The lowest value among the estimated I_{Diffi} from all stimulating electrodes (W and X in the example) is stored in the IPG 104 as I_{MinDiff}. An alternative measure, such as the Σ_{Diffi} divided by the number of return electrodes in the stimulation phase, can instead be stored as I_{MinDiff}.

In this manner, ΔV_{Ci} for the stimulating electrodes (W and X in the example) will have the same positive sign as ΔV_{dli}, as the real I_{Pi} for therapy is guaranteed to be less than I_{Ni}.

However, I_{Pi} was determined with only one electrode active. For the same I_{Pi} to flow during therapy where all programmed electrodes are active simultaneously, at least a return electrode in the stimulation phase (e.g., Z, assuming that I_{PZ} is the smallest return current amplitude of the stimulation phase) needs to be forced to present lower impedance than the sinking currents so that the I_{Ni} currents get properly established.

On the other hand, in the case of the return electrodes of the stimulation phase, except for the one forced to have lower impedance (Z in the example), the balance phase currents are preferably automatically programmed equal to

I_{Ni}=I_{Pi}−I_{MinDiff}(i=Y in the example) (10)

where was stored in the IPG 104 as described before.

The system 100 can then cycle independently through each return electrode of the stimulation phase except the forced one (only Y in the example), injecting again M (M=2, 4, 8, . . . ) pulses with the selected I_{R} and the automatically-programmed I_{Ni} (see eq. (10)) against the IPG case 201a (the return electrode in this stage).

After the M pulses, the difference between the real I_{R} and real I_{Ni} can be estimated as follows:

(real I_{Pi}−real I_{Ni})=Cᵢₘₐₓ(−ΔV_{Ci})/(MPW) (11)

The system 100 then verifies

0<(real I_{Pi}−real I_{Ni})≦I_{MinDiff} (12)

and (real I_{Pi}−real I_{Ni}) is defined as ΔIᵢ.

If condition (12) is not satisfied, the system 100 can automatically adjust I_{Ni} until condition (12) is satisfied, as I_{Pi} is the programmable parameter of the stimulation phase.

The remaining sourcing/sinking currents of the stimulation/balance phase will circulate through the forced electrode (Z in the example).

In this way, the stimulating and return electrodes charge in opposite directions, allowing for compensation when one of the conditions (2) is reached.

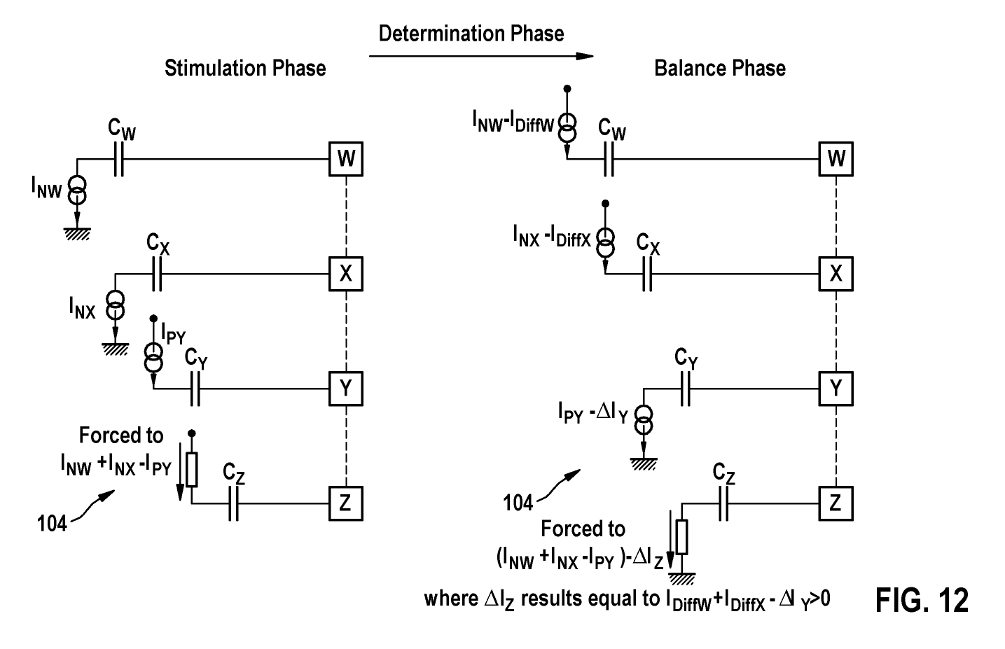

To summarize, FIG. 12 shows the stimulation and balance phases (post determination stage) for the foregoing example. Programmable resistors, instead of a current source or sink, can be used to force electrode Z (which forms the forced return electrode in the example) to present lower impedance in both the stimulation and balance phases. Considering the stimulation phase, for example, such a resistor can be programmed equal to

{V_{SDp}/I_{pZmin}+[(I_{PYmax}/I_{Pzmin})/C_{YdlYmin}−1/C_{ZdlZmax}]*PW+(I_{PYmax}/I_{Pzmin}*R_{Y2allEmax}−R_{Z2allEmin})}

where V_{SDp} is a “safe” compliance voltage required for the current sources to operate, min and max subscripts represent the respective parameters with added or subtracted errors, and R_{i2allE} (i=Y, Z in the example) is the impedance of electrode “i” against all other electrodes tied together. The selected resistance's appropriateness can be confirmed by compliance voltage monitoring across active sink and sourcing currents during the actual electrical stimulation of the target. If two or more return electrodes are programmed, electrode Z represents the electrode with the smallest programmed current.

As a final step of the determination stage, a new set of M pulses, with the determined balance phase, is preferably injected next for all active electrodes (i.e., both the stimulating and return electrodes), except for the forced one (Z in the example). The parameters ΔV_{dli} and ΔV_{Ci}|^{Per Pulse} for each electrode are now determined, the latter as the measured ΔV_{Ci}/M for the selected stimulating and return electrodes, and particularly as

[(ΣI_{Diffi}−ΣΔIᵢ)*PW]/Cᵢₘᵢₙ

for the forced electrode (Z in the example). These values are digitized and stored in the IPG 104. For the forced electrode (Z in the example), a new lookup table can be implemented to determine ΔV_{CFor}|^{Per Pulse} (the accumulated per-stimulation pulse voltage in the DC blocking capacitor associated with the forced electrode, Z in the example).

The system 100 will preferably select and monitor (during delivery of the electrical stimulation to the target) the stimulating and return electrodes that presented the largest |ΔV_{dli}|. It will also monitor the forced electrode (Z in the example). The voltages V*_{Stim}, V*_{Ret}, and V*_{For} (see FIG. 11b) of the selected stimulating, return, and forced electrodes will particularly be connected to V*_{MUXStim}, V*_{MUXRet}, and V*_{MUXFor} respectively via the MUX. The circuitry of FIG. 9/FIG. 10, except for the DAC, and the AMP of FIG. 11a are not needed for the actual electrical stimulation of the target so they may be turned off/disconnected to reduce power consumption.

In an alternative version, all voltages of the participating active electrodes may be monitored instead.

As mentioned before, during electrical stimulation of the target, the system guarantees:

ΔV_{AddOCP}≦ΔV_{dli}≦ΔV_{SubOCP}(i=1 . . . N) (13) (same as eq. (2))

Now, during an open circuit phase (where no current is imposed by the IPG 104), if the IPG case 201a is connected to V_{REF}, one has for the monitored voltages:

V_{REF}+V_{OCP}−ΔV_{dlOutput}−ΔV_{COutput}−V*_{MUXOutput}=0 (14)

(with the sign shown in FIG. 7) where Output is either Stim, Ret or For. Eq. (13) can be re-written as

ΔV_{dlOutput}=V_{REF}+V_{OCP}−ΔV_{COutput}−V*_{MUXOutput} (15)

At the same time, after P stimulation pulses,

ΔV_{COutput}=Σ_{1 to P}ΔV_{COutput}|^{Per Pulse}=PΔV_{COutput}|^{Per Pulse} (16)

where the parameter ΔV_{COutput}|^{Per Pulse} was previously digitized and internally stored in the IPG 104 in the final step of the determination stage.

Hence from (13), (15) and (16), for the monitored voltages we have

−ΔV_{AddOCP}≦V_{REF}+V_{OCP}−PΔV_{COutput}|^{Per Pulse}−V*_{MUXOutput}≦ΔV_{SubOCP} (17)

Conditions (17) can be individually re-written as

V*_{MUXStim}≧V_{REF}+V_{OCP}−ΔV_{SubOCP}−P ΔV_{CStim}|^{Per Pulse} (18.a)

V*_{MUXRet}≦V_{REF}+V_{OCP}+ΔV_{AddOCP}−P ΔV_{CRet}|^{Per Pulse} (18.b)

V*_{MUXFor}≦V_{REF}+V_{OCP}+ΔV_{AddOCP}−PΔV_{CFor}|^{Per Pulse} (18.c)

It is worth noting that ΔV_{CRet}|^{Per Pulse} and ΔV_{CFor}|^{Per Pulse} in conditions 18.b and 18.c are negative so they add to the value on the right of the foregoing inequalities.

Conditions 18 can re-written as

V*_{MUXStim}≧V_{REFStim}−PΔV_{CStim}|^{Per Pulse} (19.a)

V*_{MUXRet}≦V_{REFRet}−PΔV_{CRet}|^{Per Pulse} (19.b)

V*_{MUXFor}≦V_{REFRet}−PΔV_{CFor}|^{Per Pulse} (19.c)

where V_{REFStim} and V_{REFRet} are fixed voltages equal to (V_{REF}+V_{OCP}−ΔV_{SubOCP}) and (V_{REF}+V_{OCP}+ΔV_{AddOCP}) respectively.

In a preferred version of the system 100, condition 19.a is implemented by the comparator of FIG. 13, where an extra DAC block generates a variable reference that subtracts the stored ΔV_{CStim}|^{Per Pulse} after each stimulation phase from the internally calculated fixed voltage V_{REFStim}, for comparison following the end of the balance phase of pulse P and before the beginning of the next stimulation phase.

Similarly, conditions (19.b) and (19.c) are implemented by the comparators of FIG. 14, where a third and fourth internal DAC generate the variable comparison voltages.

If a comparator of FIG. 13 or FIG. 14 triggers (i.e., if outputs 801, 901, or 902 change logic value), after P pulses (a counter is kept in the IPG 104), the corresponding double-layer capacitance and blocking capacitor of the monitored electrode will be discharged.

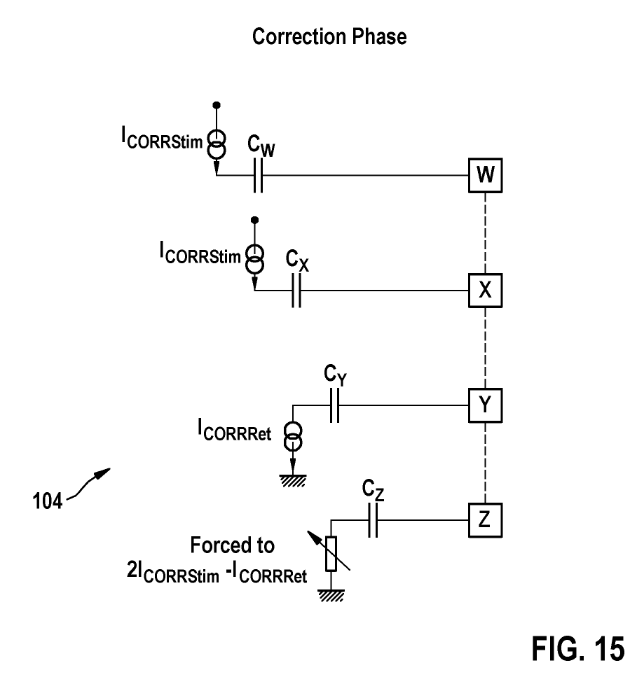

To do so, in a preferred version, a correction phase is implemented, with an example being shown in FIG. 15. A single correction current I_{CORR} will be forced to circulate, which will result in real currents I_{CORRStim} for the stimulating electrodes (W and X in the example) and I_{CORRRet} for the return electrodes (only Y in the example). The forced electrode (Z in the example) will handle the difference between the correcting currents.

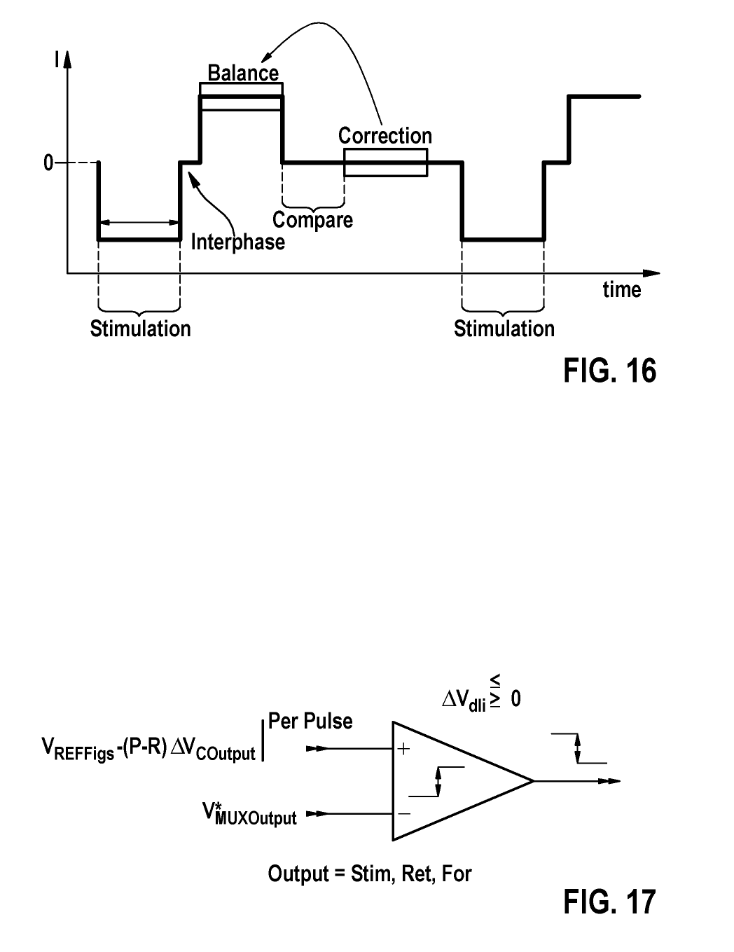

Such correction phases particularly take place following the compare phases (where conditions 18 are evaluated) as shown in FIG. 16. It is also possible to stagger the compare and correction phases so they occur in subsequent pulses. In an alternative version, the correction phase is part of the balance phase (e.g., as shown) where the currents of the balance phase are adjusted accordingly, so as to reduce or cancel the respective accumulated double layer voltage.

In a preferred version, current I_{CORR} is programmed equal to two times I_{MinDiff}.

Since it is unknown which capacitor has accumulated more charge, C_{Output} or C_{dlOutput} for the active electrode whose V*_{MUXOutput} triggered a comparator, the system 100 needs to deliver up to P pulses and stop if ΔV_{dlOutput} reaches zero voltage (ΔV_{COutput} will still be positive or negative depending on the electrode). This avoids inverting the charging conditions of the stimulating and return electrodes. Hence, during the injection of the correction phases, the system will make sure the following conditions are satisfied:

ΔV_{dlStim}=V_{REF}+V_{OCP}−ΔV_{CStim}−V*_{MUXStim}≧0 (20.a)

ΔV_{dlRet}=V_{REF}+V_{OCP}−ΔV_{CRet}−V*_{MUXRet}≦0 (20.b)

ΔV_{dlFor}=V_{REF}+V_{OCP}−ΔV_{CFor}−V*_{MUXFor}≦0 (20.c)

or re-written as

V*_{MUXStim}≦V_{REF}+V_{OCP}−ΔV_{CStim} (21.a)

V*_{MUXRet}≧V_{REF}+V_{OCP}−ΔV_{CRet} (21.b)

V*_{MUXFor}≧V_{REF}+V_{OCP}−ΔV_{CFor} (21.c)

or re-written as

V*_{MUXStim}≦V_{REFFIG5}−ΔV_{CStim} (22.a)

V*_{MUXRet}≧V_{REFFIG5}−ΔV_{CRet} (22.b)

V*_{MUXFor}≧V_{REFFIG5}−ΔV_{CFor} (22.c)

or re-written as

V*_{MUXStim}≦V_{REFFIG5}−(P−R)ΔV_{Cstim}|^{Per Pulse} (23.a)

V*_{MUXStim}≧V_{REFFIG5}−(P−R)ΔV_{CRet}|^{Per Pulse} (23.b)

V*_{MUXStim}≧V_{REFFIG5}−(P−R)ΔV_{CFor}|^{Per Pulse} (23.c)

After R correction phase pulses (R≦P), R ΔV_{COutput}|^{Per Pulse} has been subtracted from the accumulated ΔV_{COutput} (given I_{CORR} equals 2I_{MinDiff}) so V*_{MUXOutput} (of the triggered comparator) needs to be compared against a variable reference equal to V_{REFFIG5}−(P−R)ΔV_{COutput}|^{Per Pulse}, as shown in FIG. 12.

If the comparator in FIG. 17 is triggered, or R equals P, the correction phase is stopped and actual electrical stimulation of the target as per FIG. 12 resumed.

Exemplary versions of the invention have been described above in order to illustrate how to make and use the invention. The invention is not intended to be limited to these versions, but rather is intended to be limited only by the claims set out below. Thus, the invention encompasses all different versions that fall literally or equivalently within the scope of these claims.

What is claimed is:

1. A stimulation system including a pulse generator (104) having one or more electrodes (102.a, 102.b), wherein the pulse generator (104) is configured to generate:
a. a premodulated current (300) which:
(1) is output using at least one of the electrodes (102.a, 102.b),
(2) includes a train of biphasic pulses (301) having a train frequency (fₜᵣₐᵢₙ), each biphasic pulse (301) including a stimulating phase (304) and a balancing phase (305), and
b. an amplitude modulation envelope (307, 308) about the premodulated current (300), the amplitude modulation envelope (307, 308) having an envelope beat frequency (f_{beat}) smaller than the train frequency (fₜᵣₐᵢₙ) of the biphasic pulses (301).

2. The system of claim 1 wherein in each biphasic pulse (301):
a. the phases (304, 305) are rectangular pulses, and
b. the stimulating phase (304) is separated from the balancing phase (305) by an adjustable interphase delay (T_{D}).

3. The system of claim 1 wherein the modulation envelope (307, 308) of the premodulated current (300) ramps up to a maximum amplitude (I_{MAX}) and then ramps down to a minimum amplitude.

4. The system of claim 1 wherein the envelope beat frequency (f_{beat}) is automatically varied over time by at least one of:
a. the pulse generator (104), and
b. a user.

5. The system of claim 4 wherein the envelope beat frequency (f_{beat}) is varied over time by:
a. removing pulses (301) from the train over a first period, and
b. adding pulses to the train over a second period.

6. The system of claim 5 wherein pulses (301) are continuously:
a. removed from the train, and
b. added to the train,
whereby the envelope beat frequency (f_{beat}) is gradually swept between a lower envelope beat frequency (f_{beatL}) and a higher envelope beat frequency (f_{beatH}).

7. The system of claim 6 wherein the envelope beat frequency (f_{beat}) is gradually swept linearly between a lower envelope beat frequency (f_{beatL}) and a higher envelope beat frequency (f_{beatH}), whereby the variation in the envelope beat frequency (f_{beat}) over time defines a triangle wave.

8. The system of claim 1 wherein premodulated currents (300) are delivered simultaneously through several electrodes (4a, 4b, 5a, 5b, 3a, 3b), whereby current steering is effected.

9. The system of claim 1 wherein:
a. at least one of the electrodes (102.a, 102.b) defines a stimulating electrode,
b. at least one of the electrodes (102.a, 102.b) defines a return electrode,
c. each electrode is in series with:
(1) a DC blocking capacitor (Cᵢ), and
(2) a double layer capacitance (C_{dli}), wherein the double layer capacitance (C_{dli}) is defined by the electrode and material adjacent thereto;
d. the pulse generator (104) is configured to provide:
(1) a stimulation stage wherein the premodulated current (300) is output to a target using at least one of the electrodes (102.a, 102.b),
(2) a determination stage preceding the stimulation stage, wherein for each electrode through which the premodulated current (300) is output:
i. a stimulation current I_{Ni} is defined for output during the stimulating phase (304), and
ii. a balancing current I_{Pi} is defined for output during the balancing phase (305),
such that:
(a) for at least one return electrode, the difference I_{Pi}−I_{Ni} is less than or equal to the minimum of the difference I_{Pi}−I_{Ni} for all of the stimulating electrodes;
(b) for each electrode, both the DC blocking capacitor (Cᵢ) and the double layer capacitance (C_{dli}) charge in the same direction; and
(c) the stimulating electrodes charge in the opposite direction of the return electrodes.

10. The system of claim 9 wherein for each electrode, the difference I_{Pi}−I_{Ni} is a positive value.

11. The system of claim 9 wherein the pulse generator (104):
a. monitors at least one of the electrodes during the stimulation stage, and
b. applies a correction current (I_{CORRStim}, I_{CORRRet}) to each monitored electrode when a voltage (ΔV_{dli}) accumulated at the double layer of the monitored electrode crosses a pre-defined threshold (−ΔV_{AddOCP}, ΔV_{SubOCP}), wherein the correction current reduces the accumulated voltage (ΔV_{dli}).

12. A stimulation system including a pulse generator (104) having one or more electrodes (102.a, 102.b), wherein:
a. at least one of the electrodes (102.a, 102.b) defines a stimulating electrode,
b. at least one of the electrodes i (102.a, 102.b) defines a return electrode,
c. each electrode is in series with:
(1) a DC blocking capacitor (Cᵢ), and
(2) a double layer capacitance (C_{dli}), wherein the double layer capacitance (C_{dli}) is defined by the electrode and material adjacent thereto;
d. the pulse generator (104) is configured to provide:
(1) a stimulation stage wherein a premodulated current (300) is output to a target using at least one of the electrodes (102.a, 102.b), the premodulated current (300) including a train of biphasic pulses (301) having a train frequency (fₜᵣₐᵢₙ), each biphasic pulse (301) including a stimulating phase (304) and a balancing phase (305);
(2) a determination stage preceding the stimulation stage, wherein for each electrode through which the premodulated current (300) is output:
i. a stimulation current I_{Ni} is defined for output during the stimulating phase (304), and
ii. a balancing current I_{Pi} is defined for output during the balancing phase (305),
such that:
(a) for at least one return electrode, the difference I_{Pi}−I_{Ni} is less than or equal to the minimum of the difference I_{Pi}−I_{Ni} for all of the stimulating electrodes;
(b) for each electrode, both the DC blocking capacitor (Cᵢ) and the double layer capacitance (C_{dli}) charge in the same direction; and
(c) the stimulating electrodes charge in the opposite direction of the return electrodes.

13. The system of claim 12 wherein for each electrode, the difference I_{Pi}−I_{Ni} is a positive value.

14. The system of claim 12 wherein the pulse generator (104):
a. monitors at least one of the electrodes during the stimulation stage, and
b. applies a correction current (I_{CORRStim}, I_{CORRRet}) to each monitored electrode when a voltage (ΔV_{dli}) accumulated at the double layer of the monitored electrode crosses a pre-defined threshold (−ΔV_{AddOCP}, ΔV_{SubOCP}), wherein the correction current reduces the accumulated voltage (ΔV_{dli}).

15. The system of claim 12 wherein the pulse generator (104) is configured to generate an amplitude modulation envelope (307, 308) about the premodulated current (300), the amplitude modulation envelope (307, 308) having an envelope beat frequency (f_{beat}) smaller than the train frequency (fₜᵣₐᵢₙ) of the biphasic pulses (301).

16. The system of claim 15 wherein the modulation envelope (307, 308) of the premodulated current (300) ramps between a maximum amplitude (I_{MAX}) and a minimum amplitude.

17. The system of claim 15 wherein the pulse generator (104) varies the envelope beat frequency (f_{beat}) over time.

18. The system of claim 15 wherein the pulse generator (104) varies the envelope beat frequency (f_{beat}) over time by:
a. removing pulses (301) from the train over a first period, and
b. adding pulses to the train over a second period.

19. The system of claim 15 wherein wherein the pulse generator (104):
a. first continuously removes pulses (301) from the train, and
b. subsequently adds pulses (301) to the train,
whereby the envelope beat frequency (f_{beat}) is gradually swept between a lower envelope beat frequency (f_{beatL}) and a higher envelope beat frequency (f_{beatH}).

20. The system of claim 15 wherein the envelope beat frequency (f_{beat}) is gradually swept linearly between a lower envelope beat frequency (f_{beatL}) and a higher envelope beat frequency (f_{beatH}), whereby the variation in the envelope beat frequency (f_{beat}) over time defines a triangle wave.
